(19) United States
(12) Patent Application Publication
(10) Pub. No.: US 2002/0082482 A1
(43) Pub. Date: Jun. 27, 2002

(54) Member for detecting abnormality of organs and meridians and maintaining their health

(76) Inventor: Kazuo Ishii, Tokyo (JP)

Correspondence Address: HELLER EHRMAN WHITE & MCAULIFFE LLP, 1666 K STREET,NW, SUITE 300, WASHINGTON, DC, 20006, US

(21) Appl. No.: 09/984,004

(22) Filed: Oct. 26, 2001

(30) Foreign Application Priority Data
Oct. 27, 2000 (JP) 2000-328752
Dec. 21, 2000 (JP) 2000-388717

(51) Int. Cl. A61B 5/00

(52) U.S. Cl. 600/300

(57) ABSTRACT

Disclosed herein is a member for separately detecting abnormality of organs and/or meridians or maintaining their health, which contains base materials each having a color of electromagnetic waves resonant with the characteristic frequency generated when abnormality occurs at its specific organ or meridian. The color is a detection color of abnormality of the specific organ or meridian or/and its health-maintaining color.

### BACKGROUND OF THE INVENTION

1. Field of the Invention:

The present invention relates to a member for detecting abnormality of a health condition and maintaining the health condition making good use of colors, and more particularly to a member for diagnosis comprising base materials each adapted to detect abnormality of a health condition making the best use of the phenomenon that when some abnormality occurs at an organ or meridian, one's muscle-contracting force is reduced In resonance with the electromagnetic waves of its specific color, and to determine a color for health maintenance of the specific organ or meridian so as to improve the condition of the organ or meridian and achieve the effect thereof by enhancing the muscle-contracting force by the electromagnetic waves of the color for maintaining the health.

2. Description of the Background Art

The examination of a health condition has heretofore been conducted in a well-equipped hospital because the techniques of Western medicine have been developed to require high-degree examination apparatus and test drugs. Therefore, the examination can not be frequently conducted in view of the burden of time and economy on a patient. Accordingly, in many cases, the examination is conducted for the first time a patient has have a subjective symptom. As a result, in some cases, a long-term treatment may be required, or it may be too late. On the other hand, in Eastern medicine, the condition of a patient is heard, and a diagnosis is made by experiences and a sense on the basis of the hearing. Therefore, there is no need to use particularly high-degree apparatus. In the Eastern medicine, a health examination (detection of health condition) by colors has been variously attempted from of old as described in the following (1) and (2).

(1) In the Chinese medicine of the East, all phenomena (from the cosmos to human body) are classified into two opposing principles of Yin and Yang, and five elements of wood. fire, earth, metal and water. The human body includes 12 meridians (positive meridians) and the like, and the Chinese medicine has developed the five-color theory by this theory of YinYang five elements, including organs belonging to these meridians, and classified the Yin and Yang, and the five colors as shown in the following Table 1.

TABLE 1

| | Wood | Fire | Earth | Metal | Water |
|---|---|---|---|---|---|
| Yin | Liver | Heart, | Spleen | Lung | Kidney |
| meridian | | pericardium | | | |
| Yang | Gallbladder | Small | Stomach | Large | Bladder |
| meridian | | intestine, | | intestine | |
| Five | Blue | Red | Yellow | White | Black |
| colors | | | | | |

In Table 1, these 5 colors are put to practical use in a diagnosis by observing a complexion and the like of a patient, called inspection. the diagnosis is conducted by observing the complexion of the patient that appears at the time of morbidity, wherein, for example, the complexion becomes dark when the patient suffers from a kidney trouble; or the color is lost, and the complexion turns pale when the patient suffers from a lung disease. In this respect, it corresponds to the facts, and carries theoretical validity and justice. This is a reason why the inspection has maintained its life to date over a long period of time.

(2) In the ancient India, it was expounded that colors required by the human body are arranged like 7 colors (red, orange, yellow, green, blue, indigo blue and purple) of a rainbow from the genital organs to the head while developing the unique cakra theory from the time of Ayurveda.

(3) Historically, there is a diagnosis making use of muscle-contracting force of the shoulder based on applied kinesiology by Dr. Goodheart in America. As a method hinted by this, there Is a method called BDOT (Bio Digital O-ring Test) by Dr. Satoaki Omura in New York, in which an O ring is formed between the thumb and another finger to determine a difference in muscle-contracting force based on whether the ring is opened or not, Besides, there is a pull-out test in which, for example, a toothpick is held by fingers to observe whether the toothpick can be pulled out or not. These testing methods are all diagnoses of health condition called a muscle-contracting force testing method and methods for detecting the health condition. More specifically, when a person holds the same substance as a biosubstance or a substance carried by a living body, the muscle-contracting force of the person is decreased (reacted negatively) by a resonance phenomenon between both substances. The muscle-contracting force is also decreased when a harmful substance to the living body is held, and the muscle-contracting force is increased when a useful substance is held. These testing methods put these phenomena to practical use. It is considered that electromagnetic waves (wave motion) participate in this mechanism. However, sufficient clarification is not yet made.

However, such a method in Eastern medicine as described above, in which the condition of a patient is heard, and a diagnosis is made by experiences and a sense on the basis of the hearing, attaches great importance to the experiences and sense and hence involves a problem of objectivity. The above-described diagnosis (1) is a diagnosis by observing a complexion and the like of a patient called inspection. When this diagnosis is considered in the light of the muscle-contracting force testing method, it is basically correct as to the Yin meridians. However, it is entirely wrong as to the Yang meridians. When the Yin meridians are strictly considered, the blue of the liver is bluish green in the strict sense, and the yellow of the spleen Is correctly bright yellow slightly tinged with red. As described above, the Yin meridians are partially wrong. With respect to the item (2), rough points are observed in the light of the muscle-contracting force testing method. The color becomes white at the position of the diaphragm, and it was clarified that the colors are arranged in order of red, orange, yellow, green, blue, indigo blue and purple as shown collectively as the health-maintaining colors in the following Table 3. As described above, the detections by the colors have been variously attempted from of old. Those handed down until now have been tested over a long period of time and hence generally include justifiable contents, but contain wrong parts when strictly investigated Researches taking over these are also conducted, but many of them are Incorrect.

### SUMMARY OF THE INVENTION

Therefore, the present inventor has scientifically examined the whole of the five-color theory of Chinese medicine and the seven-color theory of Ayurveda In detail using the muscle-contracting force testing method while substantially taking over the valid and justifiable aspects of these theories and overcome the above-described defects and problems to specify and classify detection colors of electromagnetic waves resonant with the characteristic frequencies generated when abnormality occurs at respective specific organs or meridians, and their corresponding health-maintaining colors as to 14 meridian vessels including 2 vessels of eccentric meridians, and 28 organs. The details clarified as to the organs are shown collectively in Tables 2 and 3 which will be described subsequently This substance thus clarified has been applied to clear up a method for detecting abnormality of organs or meridians and a method for identifying the colors for maintaining the health thereof, thus leading to completion of the present invention. Therefore, It is an object of the present invention to provide a member for detecting abnormality of an organ or meridian or maintaining Its health condition, by which the health condition can be simply known on the basis of the substance extremely high in correctness, which has been obtained by being scientifically cleared up as to the five-color theory of Chinese medicine and the seven-color theory of Ayurveda using the muscle-contracting force testing method.

The above object can be achieved by the present invention described below.

According to the present invention, there is thus provided a member for separately detecting abnormality of organs and meridians or maintaining their health, which comprises base materials each having a color of electromagnetic waves resonant with the characteristic frequency generated when abnormality occurs at its specific organ or meridian, wherein the colors of the base materials satisfy the relationship shown in the following Table 2 between the colors for detecting abnormality of the respective specific meridians and their health-maintaining colors:

TABLE 2

| | | Detection | Munsell | Health- | Munsell | | Detection | Munsell | Health- | Munsell |
|---|---|---|---|---|---|---|---|---|---|---|
| | Yin | color of | color | maintaining | color | Yang | color of | color | maintaining | color |
| | meridian | abnormality | system | color | system | meridian | abnormality | system | color | system |
| Wood | Liver | Bluish green | 8.9GY | Orange (pale | 4.8YR | Gallbladder | Light gray | | Orange (pale | 4.8YR |
| | meridian | (baroque | 5.9/11.3 | golden | 5.4/11.7, | meridian | | | golden | 5.4/11.7, |
| | | green) | | brown, | 10.0R | | | | brown, | 10.0R |
| | | | | mandarin | 5.1/13.9 | | | | mandarin | 5.1/13.9 |
| | | | | orange) | | | | | orange) | |
| Fire | Heart | Red (signal | 8.0GY | Green (green | 7.8G | Small | Cream | 8.5YR | Gamboge | 3.8Y |
| | meridian | red, golden | 5.9/14.9, | lily) | 6.2/4.3 | intestine | | 8.0/3.8 | | 8.4/11.8 |
| | | red) | 8.0R | | | meridian | | | | |
| | | | 4.5/16.3 | | | | | | | |
| | Pericardium | Flesh color | 0.1Y | Pale | 0.1Y | Triple | Light purple | 2.1P | Yellowish | 5.9GY |
| | meridian | | 8.3/3.3 | yellowish | 6.9/12.4, | energizer | | 5.4/7.3 | green | 6.1/9.3 |
| | | | | orange | 6.3YR | | | | | |
| | | | | (saffron, | 6.3/11.2 | | | | | |
| | | | | nasturtium | | | | | | |
| | | | | orange) | | | | | | |
| Earth | Spleen | Gamboge | 3.8Y | White | | Stomach | Yellowish | 7.2YR | Yellow | 7.5Y |
| | meridian | | 8.4/11.8 | | | meridian | orange (light | 6.8/8.6 | (narcissus) | 8.4/11.8 |
| | | | | | | | apricot) | | | |
| Metal | Lung | White | | Bluish green | 8.9GY | Large | Beige | 5.3YR | Gamboge | 3.8Y |
| | meridian | | | (baroque | 5.9/11.3 | intestine | | 7.3/2.1 | | 8.4/11.8 |
| | | | | green) | | meridian | | | | |
| water | Kidney | Black | | Orange (pale | 4.8YR | Bladder | Dark gray | | Orange (pale | 4.8YR |
| | meridian | | | golden | 5.4/11.7, | meridian | | | golden | 5.4/11.7, |
| | | | | brown, | 10.0R | | | | brown, | 10.0R |
| | | | | mandarin | 5.1/13.9 | | | | mandarin | 5.1/13.9 |
| | | | | orange) | | | | | orange) | |
| | Governor | Pale sky | 5.68B | Vivid | 1.1GY | | | | | |
| | vessel | blue | 7.7/3.1 | purplish | 7.8/11.6 | | | | | |
| | | | | yellow (rape | | | | | | |
| | | | | color) | | | | | | |
| | Conception | Blue | 12B | Vivid bluish | 2.3G | | | | | |
| | vessel | (Turquoise | 5.6/9.5 | green | 4.2/9.3 | | | | | |
| | | blue) | | (peacock | | | | | | |
| | | | | blue) | | | | | | |

According to the present invention, there Is also provided a member for separately detecting abnormality of organs and meridians or maintaining their health, which comprises base materials each having a color of electromagnetic waves resonant with the characteristic frequency generated when abnormality occurs at its specific organ or meridian, wherein the colors of the base materials satisfy the relationship shown in the following Table 3 between the colors for detecting abnormality of the respective specific organs and their health-maintaining colors:

TABLE 3

| | Detection color of | Munsell color | Health- | Munsell color |
|---|---|---|---|---|
| Organ | abnormality | system | maintaining color | system |
| Cerebrum | Vivid purplish red | 2.6RP 3.4/13.4 | Purple (medium | 0.5RP 3.2/8.2, |
| | (fuchsia rose) | | purple, | 8.9P 3.5/10.9 |
| Cerebellum | Vivid purplish red | 2.6RP 3.4/13.4 | Purple (deep red) | 8.0R 3.3/8.9 |
| | (fuchsia rose) | | | |
| Hypophysis | Ocher (yellow gold) | 1.4Y 6.2/9.9 | Indigo blue | 0.1PB 3.8/7.6 |
| Eye | Vivid reddish | 5.0YR 5.9/12.8 | Purple (viola) | 9.6P 3.3/9.2 |
| | yellow (sun orange) | | | |
| Ear | Vivid purplish red | 2.6RP 3.4/13.4 | Purple (heavenly | 3.5RP 4.9/6.8 |
| | (fuchsia rose) | | mauve | |
| Nose | Vivid yellowish | 4.8YR 5.9/12/1 | Indigo blue | 0.1PB 3.8/7.6 |
| | orange (orange | | | |
| | peel) | | | |
| Thyroid | Rose red | 10.0RP | Blue (Turquoise | 12B 5.6/9.5 |
| gland | | 5.5/11.1 | blue) | |
| Esophagus | Orange (mandarin | 10.0R 5.1/3.9 | Beige | 5.3YR 7.3/2.1 |
| | orange) | | | |
| Thymus | Sea weed color | 2.2GY 7.2/7.5 | Red (golden red, | 8.0R 4.5/16.3, |
| | | | Red (signal red) | 8.0R 4.0/14.9 |
| Superior | Vivid yellowish red | 9.1R 4.5/13.7 | Blue (Turquoise | 12B 5.6/9..5 |
| limb | (scarlet color) | | blue) | |
| Lung | White | | Bluish green | 8.9GY 5.9/11.3 |
| | | | (baroque green) | |
| Heart | Red (golden red, | 8.0R 4.5/16.3, | Green (green | 7.8G 6.2/4.3 |
| | signal red) | 8.0R 4.0/14.9 | lily) | |
| Cardiac | Flesh color | 0.1Y 8.3/3.3 | Pale yellowish | 0.1Y 6.9/12.4 |
| valve | | | orange (saffron) | |
| Diaphragm | Baby skin pink | 2.6R 8.5/1.3 | white | |
| Stomach | Yellowish orange | 7.2YR 6.8/8.6 | Yellow | 7.5Y 8.4/11.8 |
| | (light apricot) | | (narcissus) | |
| Small | Cream | 8.5YR 8.0/3.8 | Gamboge | 3.8Y 8.4/11.8 |
| intestine | | | | |
| Large | Beige | 5.3YR 7.3/2.1 | | |
| intestine | | | | |
| Liver | Bluish green | 8.9GY 5.9/11.3 | Orange (pale | 10.0R 5.1/3.9, |
| | (baroque green) | | golden brown, | 10.0R 5.1/13.9 |
| Gallbladder | Light gray | | mandarin orange) | |
| Adrenal | Olive | 6.8Y 5.1/6.9 | | |
| Kidney | Black | | | |
| Bladder | Dark gray | | | |
| Ovary | Brown (gold Ocher) | 4.7YR 4.4/8.4 | Red (golden red, | 8.0R 4.5/16.3, |
| Uterus | Yellowish green | 8.1GY 6.5/9.3 | Red (signal red) | 8.0R 4.0/14.9 |
| | (apple green) | | | |
| Prostate | Yellowish green | 8.1GY 6.5/9.3 | | |
| | (apple green) | | | |
| Testicle | Brown (gold Ocher) | 4.7YR 4.4/8.4 | | |
| Inferior | Vivid bluish green | 2.4G 3.6/6.2 | | |
| limb | (tail green) | | | |

In the present invention, each of the base materials may have a color of electromagnetic waves resonant with the characteristic frequency generated when abnormality occurs at its specific organ or meridian, and its corresponding health-maintaining color in opposing relation to each other on the same surface of the base material.

In the present invention, each of the base materials may have, on one surface thereof, a color of electromagnetic waves resonant with the characteristic frequency generated when abnormality occurs at its specific organ or meridian, and on the other surface thereof, its corresponding health-maintaining color.

In the present invention, each of the base materials may have a rectangular form and is pivotably fixed at an end thereof.

In the present invention, the member may comprise a base material having a circular form and divided into required equal portions with the central point thereof as the central figure, and each of the portions may have a detection color of abnormality and its corresponding health-maintaining color in the Yin meridian shown in Table 2 or a detection color of abnormality and its corresponding health-maintaining color in the Yang meridian shown in Table 2.

In the present invention, the member may comprise a base material having a circular form and divided into required equal portions with the central point thereof as the central figure, and each of the portions may have a detection color of abnormality and its corresponding health-maintaining color in the organ shown in Table 3.

In the present invention, the member may comprise a base material having a rectangular form and divided into required equal portions folded zigzag.

According to the member for separately detecting abnormality of organs and meridians or maintaining their health according to an embodiment of the present invention, the base materials each have a color of electromagnetic waves resonant with the characteristic frequency generated when abnormality occurs at its specific organ or meridian, and the colors of the base materials satisfy the relationship shown in Table 2 between the colors for detecting abnormality of the respective specific meridians and their health-maintaining colors, whereby the relationship between the specific meridian and the detection color of its abnormality can be indicated on the basis of the member, and so the health condition of the meridian can be exactly diagnosed.

According to the member for separately detecting abnormality of organs and meridians or maintaining their health according to another embodiment of the present invention, the base materials each have a color of electromagnetic waves resonant with the characteristic frequency generated when abnormality occurs at its specific organ or meridian, and the colors of the base materials satisfy the relationship shown in Table 3 between the colors for detecting abnormality of the respective specific organs and their health-maintaining colors, whereby the relationship between the specific organ and the detection color of its abnormality can be indicated on the basis of the member, and so the health condition of the organ can be exactly diagnosed.

In the above-described members, each of the base materials has a color of electromagnetic waves resonant with the characteristic frequency generated when abnormality occurs at its specific organ or meridian, and its corresponding health-maintaining color in opposing relation to each other on the same surface of the base material, whereby the detection color of abnormality of the organ or meridian and its health-maintaining color can be easily recognized.

In the above-described members, each of the base materials has, on one surface thereof, a color of electromagnetic waves resonant with the characteristic frequency generated when abnormality occurs at its specific organ or meridian, and on the other surface thereof, its corresponding health-maintaining color, whereby the detection color of abnormality of the organ or meridian and its health-maintaining color can be easily recognized.

In the above-described members, each of the base materials has a rectangular form and is pivotably fixed at an end thereof, whereby the base materials can be arranged in the form of a circle with each end as a central figure, and so such a member has an effect that the selection of a color is easy.

In the above-described members, the members comprise a base material having a circular form and divided into required equal portions with the central point thereof as the central figure, and each of the portions has a detection color of abnormality and its corresponding health-maintaining color in the Yin meridian shown in Table 2 or a detection color of abnormality and its corresponding health-maintaining color in the Yang meridian shown in Table 2, whereby all colors can be provided on a small space, and the selection of a color is also easy.

In the above-described members, the members comprise a base material having a circular form and divided into required equal portions with the central point thereof as the central figure, and each of the portions has a detection color of abnormality and its corresponding health-maintaining color in the organ shown in Table 3, whereby all colors can be provided on a small space, and the selection of a color is also easy.

In the above-described members, the members comprise a base material having a rectangular form and divided into required equal portions folded zigzag, whereby the member can be store in a small space in a state folded when it is not used.

### BRIEF DESCRIPTION OF THE DRAWINGS

The above and other objects, features and advantages of the present invention will become apparent from the following description and the appended claims, taken in conjunction with the accompanying drawings, in which:

FIGS. 1A to 1D are plan views each illustrating a base material of a member for detecting abnormality of meridians or maintaining their health according to the present invention;

FIG. 2 is a plan view illustrating a member for detecting abnormality of an organ or maintaining its health according to the present invention.

FIG. 3 is a plan view illustrating a member for detecting abnormality of an organ or maintaining its health according to the present invention;

FIG. 4 is a plan view illustrating a member for detecting abnormality of organs or maintaining their health according to another embodiment of the present invention;

FIG. 5 Is a plan view illustrating a member for detecting abnormality of maintaining their health in the form of a circle according to a further embodiment of the present invention; and

FIG. 6 is a perspective view illustrating a member for detecting abnormality of organs or maintaining their health in the form of a sheet folded zigzag according to a still further embodiment of the present invention.

### DETAILED DESCRIPTION OF THE PREFERRED EMBODIMENTS

The embodiments of the present invention will hereinafter be described with reference to the drawings. However, the present invention is not limited to these embodiments.

In the present specification, the term “detection color” as used in the present invention means the specific color of electromagnetic waves resonant with the characteristic frequency generated by an abnormal meridian or organ, said abnormality being able to be detected by reduction of muscle-contracting force thereby. On the other hand, the term “health-maintaining colors” as used in the present invention means the specific color which can enhance muscle-contracting force by electromagnetic waves of the color against the characteristic frequency generated by an abnormal meridian or organ to maintain the health condition thereof. The members for separately detecting abnormality of organs and meridians or maintaining their health according to the present invention are used in diagnoses by muscle-contacting force testing methods (including an O-ring testing method). The O-ring testing method Is the above-described diagnosis developed by Dr. Satoaki Omura, in which an O ring is formed between the thumb and another finger to determine a difference in muscle-contracting force based on whether the ring is opened or not. Specifically, a subject holds a card of a yellowish orange color, which is a detection color of abnormality, by one's left hand. On the other hand, a ring is formed in an O shape by the thumb and another finger (for example, the index finger) of the right hand. During the test, the subject throws strong force into the fingers so as not to separate the contact part. The diagnostician applies one's hands to the thumb and the index finger, respectively, and pulls them in separating directions so as to separate the contact between both fingers. At this time, in the contact between the thumb and the index finger is not separated in the case where the stomach of the subject is normal even when the diagnostician pulls them in the separating directions. since the subject can throws strong force into the fingers. When the stomach of the subject is abnormal, however, the form of the O-ring cannot be maintained even when its maintenance is intended by main force, and so the contact between the thumb and the index finger Is separated. By this fact, it is known that the stomach of the subject is abnormal.

The mechanism that the abnormality of the specific meridian or organ can be detected by the color according to the O-ring testing method is not known as a whole. However, it is considered to be attributable to the fact that a morbid substance is produced, or a genetic change occurs if abnormal, and the frequency characteristic of the substance generated thereby resonates with the frequency of electromagnetic waves of the specific color, whereby a phenomenon that muscle-contracting force is reduced occurs.

This is also apparent from the following test. For example, when a 10-yen copper coin is held in secret, and another 10-yen coin is held, the muscle-contracting force of the holder is reduced because both coins have the same frequency as substances and resonate with each other, so that the O-ring is opened to find one held in secret to be a 10-yen coin. However, the muscle-contacting force is not reduced between a 10-yen copper coin and a 100-yen coin, a 1-yen aluminum coin or the like(the O-ring is not opened) because the 10-yen copper coin and the 100-yen coin or the like are different in material and do not resonate with each other at all. This fact can be confirmed by everybody by a follow-up experience or test.

The present invention has still higher reliability in the detecting method of the health conditions of the specific meridians or organs and the determination of colors required of the health maintenance of the meridians or organs than before. It has been found that the determination of these colors can be achieved in accordance with the Munsell color system commonly spread in the world. Further, the members for detecting abnormality of organs or meridians or maintaining their health have been developed on the basis of this finding. The detecting method of the health conditions of the specific meridians or organs and the determination of colors required of the health maintenance of the meridians or organs upon the preparation of the members for detecting abnormality of organs or meridians or maintaining their health will be described by the following specific examples.

Specifically, when yellowish orange on a colored paper sheet for detection is touched by the left hand of a patient who has some abnormality at one's gastric meridian or stomach as an organ, the muscle-contracting force of one's right hand is reduced, and so a ring (O-ring) formed by the thumb and another finger becomes opened though it has not been opened before the touch. In such a state, when the left hand of the patient is then caused to touch with a yellow color, the muscle-contracting force of the right hand is enhanced this time, and so the ring (O-ring) formed by the thumb and another finger remain closed. As described above, a color for enhancing the muscle-contracting force, i.e. the yellow color in this case, is determined to be a color maintaining the health condition of the gastric meridian or stomach. With respect to other meridians or organs, the respective specific colors for detecting the abnormal conditions and the specific colors for health maintenance can be found. As described above, the determination of the detection colors of abnormality of the specific meridians or organs and the colors required of the health maintenance of the meridians or organs is conducted on many subjects in accordance with the O-ring testing method, and subjects having some abnormality are further examined in an ordinary hospital. By such a method, the reliability has been able to be confirmed. The results as to the meridians are shown in Table 4.

TABLE 4

| | | Detection | Munsell | Health- | Munsell | | Detection | Munsell | Health- | Munsell |
|---|---|---|---|---|---|---|---|---|---|---|
| | Yin | color of | color | maintaining | color | Yang | color of | color | maintaining | color |
| | meridian | abnormality | system | color | system | meridian | abnormality | system | color | system |
| Wood | Liver | Bluish green | 8.9GY | Orange (pale | 4.8YR | Gallbladder | Light gray | | Orange (pale | 4.8YR |
| | meridian | (baroque | 5.9/11.3 | golden | 5.4/11.7, | meridian | | | golden | 5.4/11.7, |
| | | green) | | brown, | 10.0R | | | | brown, | 10.0R |
| | | | | mandarin | 5.1/13.9 | | | | mandarin | 5.1/13.9 |
| | | | | orange) | | | | | orange) | |
| Fire | Heart | Red (signal | 8.0GY | Green (green | 7.8G | Small | Cream | 8.5YR | Gamboge | 3.8Y |
| | meridian | red, golden | 5.9/14.9, | lily) | 6.2/4.3 | intestine | | 8.0/3.8 | | 8.4/11.8 |
| | | red) | 8.0R | | | meridian | | | | |
| | | | 4.5/16.3 | | | | | | | |
| | Pericardium | Flesh color | 0.1Y | Pale | 0.1Y | Triple | Light purple | 2.1P | Yellowish | 5.9GY |
| | meridian | | 8.3/3.3 | yellowish | 6.9/12.4, | energizer | | 5.4/7.3 | green | 6.1/9.3 |
| | | | | orange | 6.3YR | | | | | |
| | | | | (saffron, | 6.3/11.2 | | | | | |
| | | | | nasturtium | | | | | | |
| | | | | orange) | | | | | | |
| Earth | Spleen | Gamboge | 3.8Y | White | | Stomach | Yellowish | 7.2YR | Yellow | 7.5Y |
| | meridian | | 8.4/11.8 | | | meridian | orange (light | 6.8/8.6 | (narcissus) | 8.4/11.8 |
| | | | | | | | apricot) | | | |
| Metal | Lung | White | | Bluish green | 8.9GY | Large | Beige | 5.3YR | Gamboge | 3.8Y |
| | meridian | | | (baroque | 5.9/11.3 | intestine | | 7.3/2.1 | | 8.4/11.8 |
| | | | | green) | | meridian | | | | |
| Water | Kidney | Black | | Orange (pale | 4.8YR | Bladder | Dark gray | | Orange (pale | 4.8YR |
| | meridian | | | golden | 5.4/11.7, | meridian | | | golden | 5.4/11.7, |
| | | | | brown, | 10.0R | | | | brown, | 10.0R |
| | | | | mandarin | 5.1/13.9 | | | | mandarin | 5.1/13.9 |
| | | | | orange) | | | | | orange) | |
| | Governor | Pale sky | 5.68B | Vivid | 1.1GY | | | | | |
| | vessel | blue | 7.7/3.1 | purplish | 7.8/11.6 | | | | | |
| | | | | yellow (rape | | | | | | |
| | | | | color) | | | | | | |
| | Conception | Blue | 12B | Vivid bluish | 2.3G | | | | | |
| | vessel | (Turquoise | 5.6/9.5 | green | 4.2/9.3 | | | | | |
| | | blue) | | (peacock | | | | | | |
| | | | | blue) | | | | | | |

The results obtained by specifically specifying organs are shown in Table 5.

TABLE 5

| | Detection color of | Munsell color | Health- | Munsell color |
|---|---|---|---|---|
| Organ | abnormality | system | maintaining color | system |
| Cerebrum | Vivid purplish red | 2.6RP 3.4/13.4 | Purple (medium | 0.5RP 3.2/8.2, |
| | (fuchsia rose) | | purple, | 8.9P 3.5/10.9 |
| Cerebellum | Vivid purplish red | 2.6RP 3.4/13.4 | Purple (deep red) | 8.0R 3.3/8.9 |
| | (fuchsia rose) | | | |
| Hypophysis | Ocher (yellow gold) | 1.4Y 6.2/9.9 | Indigo blue | 0.1PB 3.8/7.6 |
| Eye | Vivid reddish | 5.0YR 5.9/12.8 | Purple (viola) | 9.6P 3.3/9.2 |
| | yellow (sun orange) | | | |
| Ear | Vivid purplish red | 2.6RP 3.4/13.4 | Purple (heavenly | 3.5RP 4.9/6.8 |
| | (fuchsia rose) | | mauve) | |
| Nose | Vivid yellowish | 4.8YR 5.9/12/1 | Indigo blue | 0.1PB 3.8/7.6 |
| | orange (orange | | | |
| | peel) | | | |
| Thyroid | Rose red | 10.0RP | Blue (Turquoise | 12B 5.6/9.5 |
| gland | | 5.5/11.1 | blue) | |
| Esophagus | Orange (mandarin | 10.0R 5.1/3.9 | Beige | 5.3YR 7.3/2.1 |
| | orange) | | | |
| Thymus | Sea weed color | 2.2GY 7.2/7.5 | Red (golden red, | 8.0R 4.5/16.3, |
| | | | Red (signal red) | 8.0R 4.0/14.9 |
| Superior | Vivid yellowish red | 9.1R 4.5/13.7 | Blue (Turquoise | 12B 5.6/9.5 |
| limb | (scarlet color) | | blue) | |
| Lung | White | | Bluish green | 8.9GY 5.9/11.3 |
| | | | (baroque green) | |
| Heart | Red (golden red, | 8.0R 4.5/16.3, | Green (green | 7.8G 6.2/4.3 |
| | signal red) | 8.0R 4.0/14.9 | lily) | |
| Cardiac | Flesh color | 0.1Y 8.3/3.3 | Pale yellowish | 0.1Y 6.9/12.4 |
| valve | | | orange (saffron) | |
| Diaphragm | Baby skin pink | 2.6R 8.5/1.3 | White | |
| Stomach | Yellowish orange | 7.2YR 6.8/8.6 | Yellow | 7.5Y 8.4/11.8 |
| | (light apricot) | | (narcissus) | |
| Small | Cream | 8.5YR 8.0/3.8 | Gamboge | 3.8Y 8.4/11.8 |
| intestine | | | | |
| Large | Beige | 5.3YR 7.3/2.1 | | |
| intestine | | | | |
| Liver | Bluish green | 8.9GY 5.9/11.3 | Orange (pale | 10.0R 5.1/3.9, |
| | barogue green | | golden brown, | 10.0R 5.1/13.9 |
| Gallbladder | Light gray | | mandarin orange) | |
| Adrenal | Olive | 6.8Y 5.1/6.9 | | |
| Kidney | Black | | | |
| Bladder | Dark gray | | | |
| Ovary | Brown (gold Ocher) | 4.7YR 4.4/8.4 | Red (golden red, | 8.0R 4.5/16.3. |
| Uterus | Yellowish green | 8.1GY 6.5/9.3 | Red (signal red) | 8.0R 4.0/14.9 |
| | (apple green) | | | |
| Prostate | Yellowish green | 8.1GY 6.5/9.3 | | |
| | (apple green) | | | |
| Testicle | Brown (gold Ocher) | 4.7YR 4.4/8.4 | | |
| Inferior | Vivid bluish green | 2.4G 3.6/6.2 | | |
| limb | (tail green) | | | |

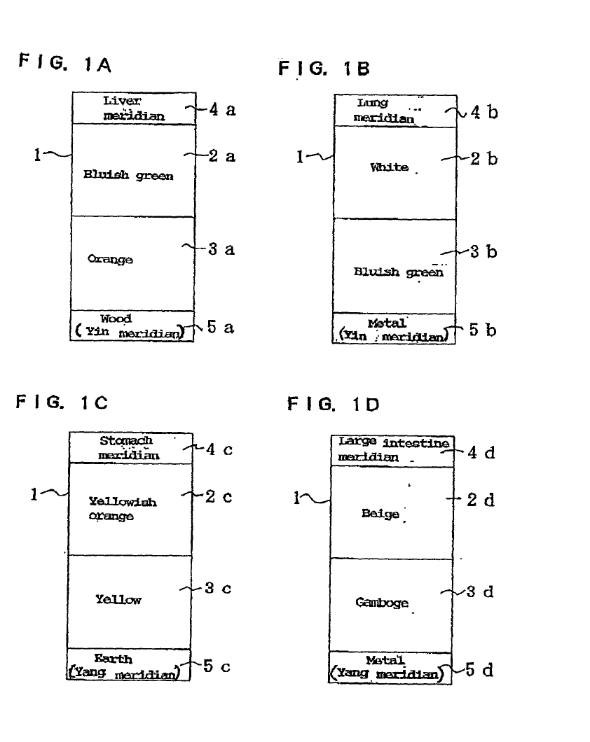

Members for separately detecting abnormality of organs and meridians or maintaining their health were prepared by applying the relationship shown in Table 4 or 5 to base materials Table 4 shows 14 meridian vessels, a horizontal row is classified into 2 poles of Yin meridian and Yang meridian, and detection colors of abnormality and health-maintaining colors are arranged therein A vertical row is classified into wood, fire, earth, metal, water and others (blank), and the horizontal rows of the Yin meridian, Yang meridian, detection colors of abnormality and health-maintaining colors, and the vertical row of the wood, fire, earth, metal, water and others (blank) are arranged in connection with each other. In Table 5, organs, detection colors of abnormality and health-maintaining colors are arranged in horizontal rows so as to correspond to respective organs in a vertical row. Members for applying these relationships to the actual O-ring testing method are specifically illustrated in FIG. 1A to FIG. 6.

The term “members for separately detecting abnormality of organs and meridians or maintaining their health” according to the present invention is used as a generic term of the members for detecting abnormality of organs or maintaining their health and the members for detecting abnormality of meridians or maintaining their health. Specific examples thereof include members for detecting abnormality or maintaining health classified with meridians as a central figure, i.e., members for detecting abnormality or health of the meridians, and members for detecting abnormality or maintaining health classified with organs as a central figure, i.e., members for detecting abnormality or health of the organs. Incidentally, the members for detecting abnormality of the meridians or maintaining their health correspond to 14 meridian vessels adding a governor vessel and a conception vessel, which are often used, among 8 vessels of eccentric meridians to the 12 meridians.

FIGS. 1A to 1D are plan views illustrating a part of a member for detecting abnormality of meridians or maintaining their health according to the present invention. In FIG. 1A, a base material for the member for detecting abnormality of meridians or maintaining their health is composed of a card-like rectangular base material 1 on the surface of which “Hepatic Meridian” 4a is printed at the upper end thereof, and “Wood (Yin Meridian)” 5a is printed at the lower end thereof. The central portion thereof is divided into two areas on the upper area 2a of which is colored with bluish green as a detection color of abnormality, and on the lower area 3a of which is colored with orange as a detection color of health (each color may be printed). In FIG. 1B, similarly “Pulmonary Meridians” 4b is printed at the upper end thereof, and “Metal (Yin Meridian)” 5b is printed at the lower end thereof The central portion thereof is divided Into two areas on the upper area 2b of which is colored with white as a detection color of abnormality, and on the lower area 3b of which is colored with bluish green as a detection color of health (each color may be printed). With respect to the Yang meridians, a member for detecting abnormality of meridians or maintaining their health is similarly prepared. More specifically, in FIG. 1C, “Gastric Meridians” 4c is printed at the upper end thereof, and “Earth (Yang Meridian)s” 5c is printed at the lower end thereof. The central portion thereof is divided into two areas on the upper area 2c of which is colored with yellowish orange as a detection color of abnormality, and on the lower area 3c of which is colored with yellow as a health-maintaining color (each color may be printed). In FIG. 1D, similarly, “Large Intestine Meridians” 4d is printed at the upper end thereof, and “Metal (Yang Meridian)” 5d is printed at the lower end thereof. The central portion thereof is divided into two areas on the upper area 2d of which is colored with beige as a detection color of abnormality, and on the lower area 3d of which is colored with gamboge as a health-maintaining color (each color may be printed). Similarly, other Yin meridians and Yang meridians than these shown in Table 2, and their relevant detection colors, health-maintaining color, etc. are provided on respective base materials to prepare a member for detecting abnormality of the meridians and maintaining their health. In the present invention, a member for detecting abnormality of organs and maintaining their health may be prepared in a similar manner to FIGS. 1A to 1D. Incidentally, in the present invention, a detection color of abnormality of an organ and its health-maintaining color may also be provided on the back surface of each of each base material of the member for detecting abnormality of the meridians and maintaining their health.

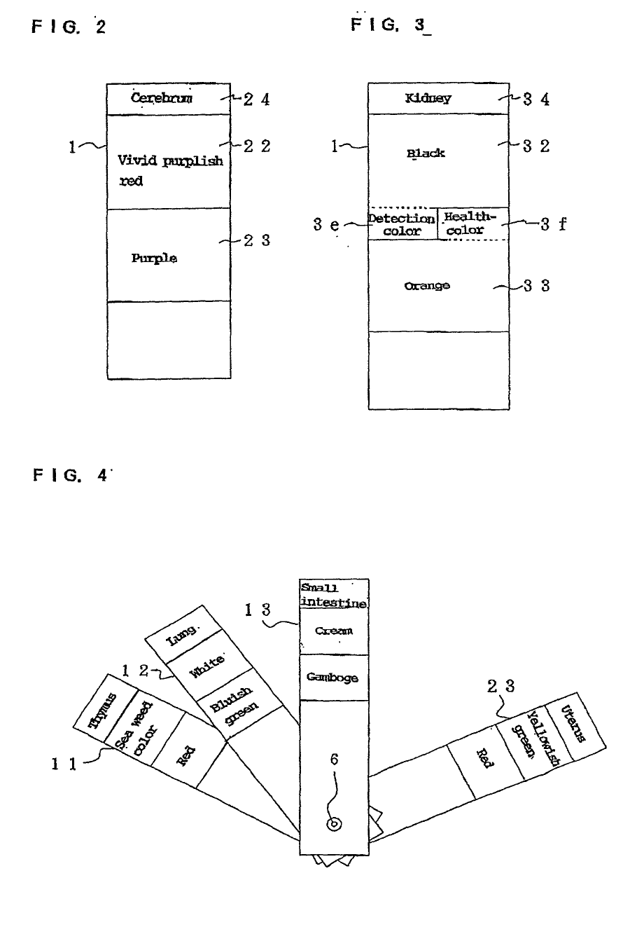

FIGS. 2 and 3 each illustrate, as a plan view, a member for detecting abnormality of an organ or maintaining its health used in the O-ring testing method prepared in accordance with the present invention. In FIG. 2, “Cerebrum” 24 is printed at the upper end on the surface of a base material 1, a blank is provided at the lower end thereof, and the central portion thereof is divided into two areas on the upper area 22 of which is colored with vivid purplish red as a detection color of abnormality, and on the lower area 23 of which is colored with purple as a health-maintaining color (each color may be printed). In FIG. 3, characters (abbreviated as “Detection Color” in FIG. 3) of a detection color of abnormality and characters (abbreviated as “Health Color” in FIG. 3) of a health-maintaining color are printed on a base material in such a manner that the detection color of abnormality and the health-maintaining color can be recognized in a moment. “Liver” 34 is printed at the upper end on the surface of the base material 1, a blank Is provided at the lower end thereof (no blank may be provided), the characters of “Detection Color” 3e and “Health Colors” 3f are printed at the central portion thereof, a blank 32 of an upper portion is colored or printed black, and a blank 33 of a lower portion is colored or printed orange.

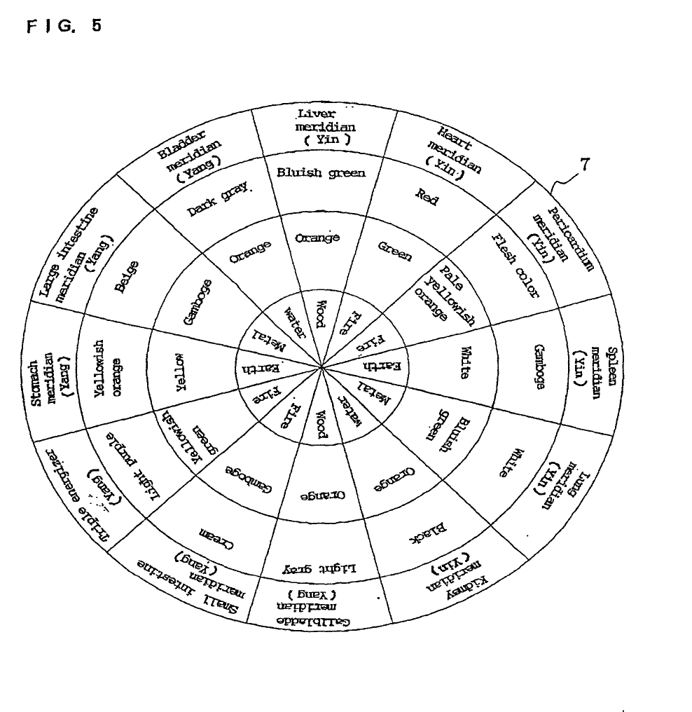

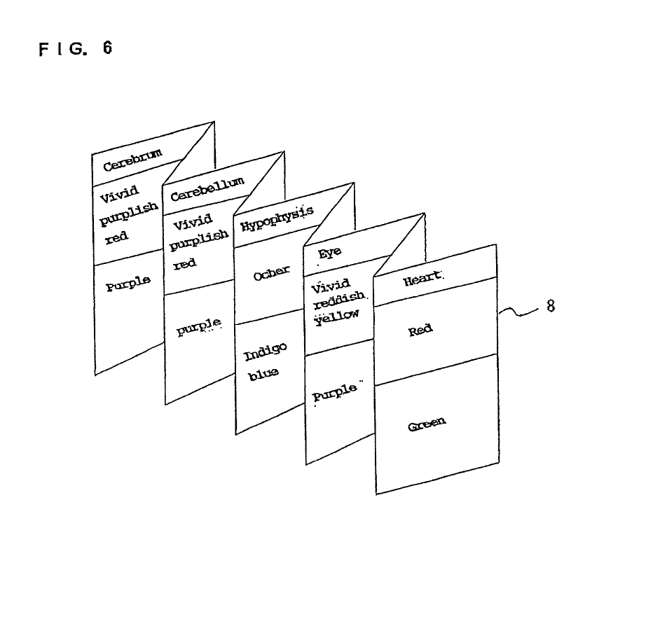

FIG. 4 is a plan view illustrating a member for detecting abnormality of organs or maintaining their health according to another embodiment of the present invention. In FIG. 4, base materials 11, 12, 13, . . . 23, . . . are provided, and for example, “Thymus”, “Lung”, “Small Intestine”, . . . “Uterus”, . . . are printed on the base materials in order from above, and detection colors of abnormality and health-maintaining colors are applied or printed on the respective base materials. These base materials 11, 12, 13, . . . 23, . . . are fixed by a rivet 6 at one ends thereof. The base materials are pivoted on the rivet 6 to open them so as to open, for example, a fun upon use. FIG. 5 is a plan view illustrating a member for detecting abnormality of meridians or maintaining their health according to a further embodiment of the present invention. In FIG. 5, a base material is in the form of a circle 7, and the surface thereof is divided into required equal portions. On each divided unit, the kind of the Yin or Yang meridian, a detection color of abnormality thereof, its corresponding health-maintaining color and an element (wood, fire, earth, metal or water) classified are printed or colored with its corresponding respective colors in that order from the peripheral part toward the center of the circle. FIG. 6 is a perspective view illustrating a member for detecting abnormality of organs or maintaining their health according to a still further embodiment of the present invention. In this embodiment, a base material 8 is in the form of a sheet folded zigzag, and the relationship between the organs and the colors as shown in Table 5 is printed or colored with respective colors corresponding to the organs. In FIG. 6, the surface of the rectangular base material is divided into required equal portions. On each divided unit, the name (for example, Cerebrum) of an organ, a detection color of abnormality thereof and its corresponding health-maintaining color are printed or colored with its corresponding respective colors in that order from above. The base materials is then folded zigzag, and a necessary portion is opened upon use. From the viewpoint of making the member for detecting abnormality of organs or maintaining their health compact, on the back surface thereof, a part of the relationship between the organs and the colors as shown in Table 5 may be printed or colored with respective colors corresponding to the organs. This has an effect of easy use. The members for detecting abnormality of organs or maintaining their health according to the present invention have been described in detail with respect to FIGS. 2 to 5 and FIG. 6. However, the members for detecting abnormality of meridians or maintaining their health according to the present invention may be prepared in a similar manner. Incidentally, in the present invention, the relationship between the meridians and the colors as shown in Table 4 may be printed or colored with respective colors corresponding to the meridians on the back surface of each of the members for detecting abnormality of the organs or maintaining their health.

As described above, in the present invention, any form such as a rectangle, circle, oval or hexagon may be used as the form of the members for detecting abnormality of the organs and/or meridians or maintaining their health. However, the forms shown in FIGS. 1 to 6 are preferred in view of the relationship shown in Tables 4 and 5. No particular limitation is imposed on the material of the base materials used In the members for detecting abnormality of the organs and/or meridians or maintaining their health according to the present invention. However, examples thereof include paper, cardboards, fabrics (woven fabrics, nonwoven fabrics, etc.), plastics, wood plates, glass sheets, metal plates and ceramic plates. Incidentally, Table 6 set forth in the appended claims is the same as Tables 2 and 4 in the description of the present invention described above, while Table 7 set forth in the appended claims is the same as Tables 3 and 5.

According to the present invention, as described above, there can be provided members for detecting abnormality of organs and/or meridians or maintaining their health, comprising base materials each having a color of electromagnetic waves resonant with the characteristic frequency generated when abnormality occurs at its specific organ or meridian, said color being a detection color of abnormality of the specific organ or meridian or/and Its health-maintaining color, by which the health condition of the organ can be easily diagnosed on the basis of the substance of the five-color theory of Chinese medicine and the seven-color theory of Ayurveda cleared up using the muscle-contracting force testing method. In the present invention, a color of electromagnetic waves resonant with the characteristic frequency generated when abnormality occurs at its specific organ or meridian, and its corresponding health-maintaining color are provided in opposing relation to each other on the same surface of the base material, whereby the relationship between the detection color of abnormality and its corresponding health-maintaining color can be clearly known to maintain the health of the organ or meridian using the health-maintaining color.

In the present invention, each of the base materials is in the form of a rectangle, and is pivotably fixed at an end thereof, whereby the base materials can be opened in a moment so as to open a fun and prevented from being separated from one another, and so such a member is easy to use. In the present invention, the member may comprises a base material having a circular form and divided into required equal portions with the central point thereof as the central figure, and each of the portions has a detection color of abnormality and its corresponding health-maintaining color in the Yin meridian shown in Table 2 or a detection color of abnormality and its corresponding health-maintaining color in the Yang meridian shown in Table 2, whereby the selection of a color is easy, and the health-maintaining color can be known in a moment as a result of the test. In the present invention, the member may comprise a base material having a rectangular form and divided Into required equal portions folded zigzag, whereby the member can be folded in a small size, and required numbers of diagnosis items can be indicated in a lot on the front and back surfaces thereof to bring about an effect of easy use.

What is claimed is:

1. A member for separately detecting abnormality of organs and meridians or maintaining their health, which comprises base materials each having a color of electromagnetic waves resonant with the characteristic frequency generated when abnormality occurs at its specific organ or meridian, wherein the colors of the base materials satisfy the relationship shown in the following Table 6 between the colors for detecting abnormality of the respective specific meridians and their health-maintaining colors:
TABLE 6

| | | Detection | Munsell | Health- | Munsell | | Detection | Munsell | Health- | Munsell |
|---|---|---|---|---|---|---|---|---|---|---|
| | Yin | color of | color | maintaining | color | Yang | color of | color | maintaining | color |
| | meridian | abnormality | system | color | system | meridian | abnormality | system | color | system |
| Wood | Liver | Bluish green | 8.9GY | Orange (pale | 4.8YR | Gallbladder | Light gray | | Orange (pale | 4.8YR |
| | meridian | (baroque | 5.9/11.3 | golden | 5.4/11.7, | meridian | | | golden | 5.4/11.7, |
| | | green) | | brown, | 10.0R | | | | brown, | 10.0R |
| | | | | mandarin | 5.1/13.9 | | | | mandarin | 5.1/13.9 |
| | | | | orange) | | | | | orange) | |
| Fire | Heart | Red (signal | 8.0GY | Green (green | 7.8G | Small | Cream | 8.5YR | Gamboge | 3.8Y |
| | meridian | red, golden | 5.9/14.9, | lily) | 6.2/4.3 | intestine | | 8.0/3.8 | | 8.4/11.8 |
| | | red) | 8.0R | | | meridian | | | | |
| | | | 4.5/16.3 | | | | | | | |
| | Pericardium | Flesh color | 0.1Y | Pale | 0.1Y | Triple | Light purple | 2.1P | Yellowish | 5.9GY |
| | meridian | | 8.3/3.3 | yellowish | 6.9/12.4, | energizer | | 5.4/7.3 | green | 6.1/9.3 |
| | | | | orange | 6.3YR | | | | | |
| | | | | (saffron, | 6.3/11.2 | | | | | |
| | | | | nasturtium | | | | | | |
| | | | | orange) | | | | | | |
| Earth | Spleen | Gamboge | 3.8Y | White | | Stomach | Yellowish | 7.2YR | Yellow | 7.5Y |
| | meridian | | 8.4/11.8 | | | meridian | orange (light | 6.8/8.6 | (narcissus) | 8.4/11.8 |
| | | | | | | | apricot) | | | |
| Metal | Lung | White | | Bluish green | 8.9GY | Large | Beige | 5.3YR | Gamboge | 3.8Y |
| | meridian | | | (baroque | 5.9/11.3 | intestine | | 7.3/2.1 | | 8.4/11.8 |
| | | | | green) | | meridian | | | | |
| Water | Kidney | Black | | Orange (pale | 4.8YR | Bladder | Dark gray | | Orange (pale | 4.8YR |
| | meridian | | | golden | 5.4/11.7, | meridian | | | golden | 5.4/11.7, |
| | | | | brown, | 10.0R | | | | brown, | 10.0R |
| | | | | mandarin | 5.1/13.9 | | | | mandarin | 5.1/13.9 |
| | | | | orange) | | | | | orange) | |
| | Governor | Pale sky | 5.68B | Vivid | 1.1GY | | | | | |
| | vessel | blue | 7.7/3.1 | purplish | 7.8/11.6 | | | | | |
| | | | | yellow (rape | | | | | | |
| | | | | color) | | | | | | |
| | Conception | Blue | 12B | Vivid bluish | 2.3G | | | | | |
| | vessel | (Turquoise | 5.6/9.5 | green | 4.2/9.3 | | | | | |
| | | blue) | | (peacock | | | | | | |
| | | | | blue) | | | | | | |

2. A member for separately detecting abnormality of organs and meridians or maintaining their health, which comprises base materials each having a color of electromagnetic waves resonant with the characteristic frequency generated when abnormality occurs at its specific organ or meridian, wherein the colors of the base materials satisfy the relationship shown in the following Table 7 between the colors for detecting abnormality of the respective specific organs and their health-maintaining colors:
TABLE 7

| | Detection color of | Munsell color | Health- | Munsell color |
|---|---|---|---|---|
| Organ | abnormality | system | maintaining color | system |
| Cerebrum | Vivid purplish red | 2.6RP 3.4/13.4 | Purple (medium | 0.5RP 3.2/8.2, |
| | (fuchsia rose) | | purple, | 8.9P 3.5/10.9 |
| Cerebellum | Vivid purplish red | 2.6RP 3.4/13.4 | Purple (deep red) | 8.0R 3.3/8.9 |
| | (fuchsia rose) | | | |
| Hypophysis | Ocher (yellow gold) | 1.4Y 6.2/9.9 | Indigo blue | 0.1PB 3.8/7.6 |
| Eye | Vivid reddish | 5.0YR 5.9/12.8 | Purple (viola) | 9.6P 3.3/9.2 |
| | yellow (sun orange) | | | |
| Ear | Vivid purplish red | 2.6RP 3.4/13.4 | Purple (heavenly | 3.5RP 4.9/6.8 |
| | (fuchsia rose) | | mauve) | |
| Nose | Vivid yellowish | 4.8YR 5.9/12/1 | Indigo blue | 0.1PB 3.8/7.6 |
| | orange (orange | | | |
| | peel) | | | |
| Thyroid | Rose red | 10.0RP | Blue (Turquoise | 12B 5.6/9.5 |
| gland | | 5.5/11.1 | blue) | |
| Esophagus | Orange (mandarin | 10.0R 5.1/3.9 | Beige | 5.3YR 7.3/2.1 |
| | orange) | | | |
| Thymus | Sea weed color | 2.2GY 7.2/7.5 | Red (golden red, | 8.0R 4.5/16.3, |
| | | | Red (signal red) | 8.0R 4.0/14.9 |
| Superior | Vivid yellowish red | 9.1R 4.5/13.7 | Blue (Turquoise | 12B 5.6/9.5 |
| limb | (scarlet color) | | blue) | |
| Lung | White | | Bluish green | 8.9GY 5.9/11.3 |
| | | | (baroque green) | |
| Heart | Red (golden red, | 8.0R 4.5/16.3, | Green (green | 7.8G 6.2/4.3 |
| | signal red) | 8.0R 4.0/14.9 | lily) | |
| Cardiac | Flesh color | 0.1Y 8.3/3.3 | Pale yellowish | 0.1Y 6.9/12.4 |
| valve | | | orange (saffron) | |
| Diaphragm | Baby skin pink | 2.6R 8.5/1.3 | white | |
| Stomach | Yellowish orange | 7.2YR 6.8/8.6 | Yellow | 7.5Y 8.4/11.8 |
| | (light apricot) | | (narcissus) | |
| Small | Cream | 8.5YR 8.0/3.8 | Gamboge | 3.8Y 8.4/11.8 |
| intestine | | | | |
| Large | Beige | 5.3YR 7.3/2.1 | | |
| intestine | | | | |
| Liver | Bluish green | 8.9GY 5.9/11.3 | Orange (pale | 10.0R 5.1/3.9, |
| | (baroque green) | | golden brown | 10.0R 5.1/13.9 |
| Gallbladder | Light gray | | mandarin orange) | |
| Adrenal | Olive | 6.8Y 5.1/6.9 | | |
| Kidney | Black | | | |
| Bladder | Dark gray | | | |
| Ovary | Brown (gold Ocher) | 4.7YR 4.4/8.4 | Red (golden red, | 8.0R 4.5/16.3, |
| Uterus | Yellowish green | 8.1GY 6.5/9.3 | Red (signal red) | 8.0R 4.0/14.9 |
| | (apple green) | | | |
| Prostate | Yellowish green | 8.1GY 6.5/9.3 | | |
| | (apple green) | | | |
| Testicle | Brown (gold Ocher) | 4.7YR 4.4/8.4 | | |
| Inferior | Vivid bluish green | 2.4G 3.6/6.2 | | |
| limb | (tail green) | | | |

3. The member according to claim 1 or 2, wherein each of the base materials has a color of electromagnetic waves resonant with the characteristic frequency generated when abnormality occurs at its specific organ or meridian, and its corresponding health-maintaining color in opposing relation to each other on the same surface of the base material.

4. The member according to claim 1 or 2, wherein each of the base materials has, on one surface thereof, a color of electromagnetic waves resonant with the characteristic frequency generated when abnormality occurs at its specific organ or meridian, and on the other surface thereof, its corresponding health-maintaining color.

5. The member according to any one of claims 1 to 4. wherein each of the base materials has a rectangular form and is pivotably fixed at an end thereof.

6. The member according to claim 1 or 3. wherein the member comprises a base material having a circular form and divided into required equal portions with the central point thereof as the central figure, and each of the portions has a detection color of abnormality and its corresponding health-maintaining color in the Yin meridian shown in Table 6 or a detection color of abnormality and its corresponding health-maintaining color in the Yang meridian shown in Table 6.

7. The member according to claim 2 or 3, wherein the member comprises a base material having a circular form and divided into required equal portions with the central point thereof as the central figure, and each of the portions has a detection color of abnormality and its corresponding health-maintaining color in the organ shown in Table 7.

8. The member according to claim 6 or 7, wherein the member comprises a base material having a rectangular form and divided into required equal portions folded zigzag.
